(19) United States
(12) Patent Application Publication
(10) Pub. No.: US 2020/0239386 A1
(43) Pub. Date: Jul. 30, 2020

(54) SYSTEM AND METHOD FOR DEHYDROGENATING ISOBUTANE TO ISOBUTYLENE

(71) Applicant: SABIC Global Technologies B.V., Bergen op Zoom (NL)

(72) Inventors: Faysal BENASKAR, Jubail (SA); Sami AL-MUTAIRI, Jubail (SA); Massimiliano COMOTTI, Jubail (SA); Adel AL-SHAFAI, Jubail (SA); Abdullah AL-JEAAN, Jubail (SA); Radhi AL-SHAMMARI, Jubail (SA)

(21) Appl. No.: 16/634,919

(22) PCT Filed: Jul. 31, 2018
(86) PCT No.: PCT/IB2018/055723

§ 371 Date: Jan. 29, 2020

Related U.S. Application Data
(60) Provisional application No. 62/539,390, filed on Jul. 31, 2017.

(51) Int. Cl. C07C 5/333; C07C 41/06; B01J 8/04; B01J 8/02

(52) U.S. Cl. CPC C07C 5/3337; C07C 41/06; B01J 8/0453; B01J 8/0292; C07C 11/09; B01J 2208/00539; B01J 2208/025; B01J 2208/00938; B01J 2208/00513

(57) ABSTRACT

A system and a method for dehydrogenating isobutane to isobutylene are disclosed. The system comprises a fixed bed dehydrogenation reactor. The fixed reactor bed in the fixed bed dehydrogenation reactor includes a catalyst layer, a first material adapted to improve the flow distribution in the fixed reactor bed, a second material adapted to improve the thermal distribution in the fixed reactor bed, and a third material adapted to improve both the flow distribution and the thermal distribution in the fixed reactor bed. The first material covers a top, a bottom, and at least a portion of a side surface of the catalyst layer of the fixed reactor bed. The second material and the third material both are evenly distributed in the catalyst layer.

### CROSS REFERENCE TO RELATED APPLICATIONS

This application claims the benefit of priority of U.S. Provisional Patent Application No. 62/539,390, filed Jul. 31, 2017, which is hereby incorporated by reference in its entirety.

### FIELD OF INVENTION

The present invention generally relates to alkane dehydrogenation processes. More specifically, the present invention relates to dehydrogenating isobutane with a fixed reactor bed that has improved flow distribution, improved heat distribution, and heat storage capabilities.

### BACKGROUND OF THE INVENTION

Isobutylene is an intermediate product for the production of methyl-tertiary-butyl ether (MTBE), which is one of the most commonly used octane boosters for gasoline. Typically, isobutylene is produced by on-purpose dehydrogenation of isobutane in a fixed bed dehydrogenation reactor. In the dehydrogenation process, the feed stream comprising isobutane flows through the fixed catalyst bed of the fixed bed dehydrogenation reactor to form isobutylene and hydrogen. During a reaction run, the catalyst of the fixed catalyst bed is gradually deactivated by coke formed on the active sites of the catalyst and the temperature of the fixed catalyst bed continuously decreases as the endothermic dehydrogenation reaction absorbs heat from the fixed catalyst bed. Therefore, at the end of the reaction run, the fixed bed dehydrogenation reactor is purged and the fixed catalyst bed is reheated and regenerated to remove the coke formed thereon and restore the temperature for the fixed catalyst bed.

Conventional fixed catalyst bed in a dehydrogenation reactor typically includes a layer of flow distribution material disposed on the top and at the bottom of a catalyst layer (chemical converting material). The catalyst layer is generally parallel to the flow distribution material. A heat distribution layer comprising heat distribution material is disposed in the middle of the catalyst layer, parallel to both flow distribution material and the catalyst layer. Overall, there are a few issues weighing against the efficiency of the conventional fixed bed hydrogenation reactor. First, the air/oxygen used to regenerate/reheat the fixed catalyst bed flows to the catalyst from the top of the fixed catalyst bed. With the heat distribution layer disposed in the middle of the catalyst layer and flow distribution layer merely laying on top and bottom surface of the catalyst layer, heat carried by the air/oxygen cannot be uniformly distributed throughout the fixed catalyst bed, resulting in uneven heating of the fixed catalyst bed. The non-uniform heat distribution in the fixed catalyst bed increases the coke formation on the catalyst and reduces catalyst life. Secondly, the flow distribution of the hydrocarbon (including isobutane) within the fixed catalyst bed is limited due to the flow distribution layers merely laying on the top and the bottom of the catalyst layer, thereby causing high pressure drop along the length of the fixed catalyst bed. Overall, the reaction efficiency and hydrocarbon conversion rate are relatively low for conventional fixed bed dehydrogenation reactors. Improvements in this field are needed.

### BRIEF SUMMARY OF THE INVENTION

A method has been discovered for dehydrogenating isobutane at an improved isobutane to isobutylene conversion rate. By reacting isobutane over a fixed reactor bed that comprises a first material adapted to improve flow distribution, a second material adapted to improve heat distribution, and a third material adapted to improve both flow distribution and heat distribution, the temperature of the fixed catalyst bed is more uniform and the isobutane/isobutylene flow is more evenly distributed through the fixed catalyst bed. Hence, the coke formation on the catalyst can be reduced and the catalyst life can be increased, thereby reducing operating cost for dehydrogenating isobutane.

Embodiments of the invention include a method of dehydrogenating isobutane (C₄H₁₀) to isobutylene (C₄H₈). The method includes the steps of flowing a hydrocarbon feed stream comprising the isobutane through a fixed reactor bed under reaction conditions sufficient to dehydrogenate the isobutane to the isobutylene. The fixed reactor bed includes a catalyst adapted to accelerate dehydrogenation of the isobutane to the isobutylene; a first material adapted to improve flow distribution such that a time difference for the hydrocarbon feed stream flowing between a center and an edge of the reactor at a planar cross-section is in a range of 0.1 to 10 seconds; and a second material adapted to improve heat distribution such that a difference between a temperature at a first location and a temperature at a second location in the reactor bed does not exceed 60° C., and flowing the isobutylene from the fixed reactor bed.

Embodiments of the invention include a method of dehydrogenating isobutane (C₄H₁₀) to isobutylene (C₄H₈). The method includes flowing a hydrocarbon feed stream comprising the isobutane through a fixed reactor bed under reaction conditions sufficient to dehydrogenate the isobutane to the isobutylene. The fixed reactor bed includes a catalyst adapted to accelerate dehydrogenation of the isobutane to the isobutylene. The fixed reactor bed further includes a first material adapted to improve flow distribution such that a time difference for the hydrocarbon feed stream flowing between a center and an edge of the reactor at a planar cross-section is in a range of 0.1 to 10 seconds. The fixed reactor bed further includes a second material adapted to improve heat distribution such that a difference between a temperature at a first location and a temperature at a second location in the reactor bed does not exceed 50° C. The fixed reactor bed further still includes a third material that is inert with respect to the dehydrogenating of the isobutane to the isobutylene. The method further includes flowing the isobutylene from the fixed reactor bed.

Embodiments of the invention include a method of dehydrogenating isobutane (C₄H₁₀) to isobutylene (C₄H₈). The method includes flowing a hydrocarbon feed stream comprising the isobutane through a fixed reactor bed under reaction conditions sufficient to dehydrogenate the isobutane to the isobutylene. The fixed reactor bed includes a chromium-based catalyst adapted to accelerate dehydrogenation of the isobutane to isobutylene. The fixed reactor bed further includes a first material adapted to improve flow distribution such that a time difference for the hydrocarbon feed stream flowing between a center and an edge of the reactor at a planar cross-section is in a range of 0.1 to 10 seconds. The fixed reactor bed further includes a second material adapted to improve heat distribution such that a difference between a temperature at a first location and a temperature at a second location in the reactor bed does not exceed 50° C. The fixed reactor bed further still includes a third material that is inert with respect to the dehydrogenating of the isobutane to the isobutylene. The method further includes flowing the isobutylene from the fixed reactor bed. The method further still includes reacting the isobutylene with methanol to form methyl-tertiary-butyl ether (MTBE).

Embodiments of the invention include a fixed bed reactor for dehydrogenating a hydrocarbon. The fixed bed reactor includes a reactor shell. The fixed bed reactor further includes a fixed reactor bed disposed in the reactor shell. The fixed reactor bed includes a catalyst adapted to accelerate dehydrogenation of the isobutane to the isobutylene. The fixed reactor bed further includes a first material adapted to improve flow distribution such that a time difference for the hydrocarbon feed stream flowing between a center and an edge of the reactor at a planar cross-section is in a range of 0.1 to 10 seconds. The fixed reactor bed further includes a second material adapted to improve heat distribution such that a difference between a temperature first location and a temperature at a second location in the reactor bed does not exceed 50° C. The fixed reactor bed further still includes and a third material that is inert with respect to the dehydrogenating of the isobutane to the isobutylene. The fixed bed reactor further includes a hydrocarbon inlet disposed on the reactor shell, wherein the hydrocarbon inlet is adapted to receive a hydrocarbon feed stream and/or a regeneration gas into the reactor shell. The fixed bed reactor further still includes an outlet disposed on an opposite side of the reactor shell to the hydrocarbon inlet, wherein the outlet is adapted to discharge a product stream from the reactor shell.

The following includes definitions of various terms and phrases used throughout this specification.

The terms “about” or “approximately” are defined as being close to as understood by one of ordinary skill in the art. In one non-limiting embodiment the terms are defined to be within 10%, preferably, within 5%, more preferably, within 1%, and most preferably, within 0.5%.

The terms “wt. %”, “vol. %” or “mol. %” refers to a weight, volume, or molar percentage of a component, respectively, based on the total weight, the total volume, or the total moles of material that includes the component. In a non-limiting example, 10 moles of component in 100 moles of the material is 10 mol. % of component.

The term “substantially” and its variations are defined to include ranges within 10%, within 5%, within 1%, or within 0.5%.

The terms “inhibiting” or “reducing” or “preventing” or “avoiding” or any variation of these terms, when used in the claims and/or the specification, includes any measurable decrease or complete inhibition to achieve a desired result.

The term “effective,” as that term is used in the specification and/or claims, means adequate to accomplish a desired, expected, or intended result.

The term “thermally stable” as that term is used in the specification and/or claims means remaining chemically and physically unchanged, at least, within a temperature range of 500 to 750° C.

The use of the words “a” or “an” when used in conjunction with the term “comprising,” “including,” “containing,” or “having” in the claims or the specification may mean “one,” but it is also consistent with the meaning of “one or more,” “at least one,” and “one or more than one.”

The words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The process of the present invention can “comprise,” “consist essentially of,” or “consist of” particular ingredients, components, compositions, etc., disclosed throughout the specification.

In the context of the present invention, at least twenty embodiments are now described. Embodiment 1 is a method of dehydrogenating isobutane (C₄H₁₀) to isobutylene (C₄H₈). The method includes the steps of flowing a hydrocarbon feed stream containing the isobutane through a fixed reactor bed under reaction conditions sufficient to dehydrogenate the isobutane to the isobutylene, wherein the fixed reactor bed includes a catalyst adapted to accelerate dehydrogenation of the isobutane to the isobutylene; a first material adapted to improve flow distribution such that a time difference for the hydrocarbon feed stream flowing between a center and an edge of the reactor at a planar cross-section is in a range of 0.1 to 10 seconds; a second material adapted to improve heat distribution such that a difference between a temperature at a first location and a temperature at a second location in the reactor bed does not exceed 60° C.; and a third material that is inert with respect to the dehydrogenating of the isobutane to the isobutylene; and flowing the isobutylene from the fixed reactor bed. Embodiment 2 is the method of embodiment 1, further comprising reacting the isobutylene with methanol to form methyl tertiary butyl ether (MTBE). Embodiment 3 is the method of any of embodiments 1 and 2, wherein the reaction conditions include a reaction temperature in a range of 500 to 700° C. Embodiment 4 is the method of any of embodiments 1 to 3, wherein the reaction conditions include a reaction pressure in a range of 0.02 to 0.9 bar. Embodiment 5 is the method of any of embodiments 1 to 4, wherein the catalyst is selected from the group consisting of chromium oxide, platinum, platinum-tin, and combinations thereof. Embodiment 6 is the method of any of embodiments 1 to 5, wherein the time difference for the hydrocarbon feed stream flowing between the center and the edge of the reactor at a planar cross-section is in a range of 1 to 5 seconds. Embodiment 7 is the method of any of embodiments 1 to 6, wherein the first material adapted to improve flow distribution comprises a solid thermally stable inert material premixed with the second material and the third materials in the fixed reactor bed. Embodiment 8 is the method of embodiment 7, wherein the thermally stable inert material is selected from the group consisting of oxides or carbides of Al, Si, Ti, Zr, Zn, Ce, Mg, Ca, La, Cs, Ba, and combinations thereof. Embodiment 9 is the method of any of embodiments 1 to 8, wherein the first material has a particle size of 5 to 35 mm and a geometric shape of substantially spherical (shape). Embodiment 10 is the method of any of embodiments 1 to 9, wherein the first material has a thermal conductivity in a range of 0.05 to 5 W/m/K, and an absolute porosity of 0 to 0.3(−). Embodiment 11 is the method of any of embodiments 1 to 10, wherein the second material adapted to improve heat distribution comprises a conductive material and/or an insulating material. Embodiment 12 is the method of embodiment 11, wherein the conductive material is selected from the group consisting of metal or oxides of Al, Si, Ti, Zr, Zn, Ce, Mg, Ca, La, Cu, Au, Sn, Fe, W, Ni, Co, Cs, Ba, alloys thereof, and combinations thereof and the insulating material is selected from the group consisting of oxides or carbides of Al, Si, Ti, Zr, Zn, Ce, Mg, Ca, La, Cs, Ba, and combinations thereof. Embodiment 13 is the method of any of embodiments 1 to 12, wherein the second material is adapted to maintain a temperature drop thereof less than 40° C. within at least 8 minutes. Embodiment 14 is the method of any of embodiments 1 to 13, wherein the second material has a particle size of 2 to 15 mm and a geometric shape of substantially cylindrical (shape). Embodiment 15 is the method of any of embodiments 1 to 14, wherein the second material has a thermal conductivity in a range of 0.4 to 200 W/m/K, and an absolute porosity of 0 to 0.5(−). Embodiment 16 is the method of any of embodiments 1 to 15, wherein the third material contains a non-reactive material that is adapted to increase flow distribution and heat distribution in the fixed reactor bed. Embodiment 17 is the method of embodiment 16, wherein the third material includes oxides or carbides of Al, Si, Ti, Zr, Zn, Ce, Mg, Ca, La, Cs, Ba, or combinations thereof. Embodiment 18 is the method of any of embodiments 1 to 17, wherein the isobutane has a conversion rate of 45 to 60%. Embodiment 19 is the method of any of embodiments 1 to 18, wherein the flowing the hydrocarbon feed stream does not include injecting sulfur to the fixed reactor bed.

Embodiment 20 is a fixed bed reactor for dehydrogenating a hydrocarbon. The fixed bed reactor includes a reactor shell; a fixed reactor bed comprising a catalyst adapted to accelerate dehydrogenation of the isobutane to the isobutylene; a first material adapted to improve flow distribution such that a time difference for the hydrocarbon feed stream flowing between a center and an edge of the reactor at a planar cross-section is in a range of 0.1 to 10 seconds a second material adapted to improve heat distribution such that a difference between a temperature at a first location and a temperature at a second location in the reactor bed does not exceed 60° C.; and third material that is inert with respect to the dehydrogenating of the isobutane to the isobutylene; a hydrocarbon inlet disposed on a side of the reactor shell, wherein the hydrocarbon inlet is adapted to receive a hydrocarbon feed stream and/or a regeneration gas into the reactor shell; and an outlet disposed on a side of the reactor shell that is opposite to the side that hydrocarbon inlet is disposed on, wherein the outlet is adapted to discharge a product stream from the reactor shell.

Other objects, features and advantages of the present invention will become apparent from the following figures, detailed description, and examples. It should be understood, however, that the figures, detailed description, and examples, while indicating specific embodiments of the invention, are given by way of illustration only and are not meant to be limiting. Additionally, it is contemplated that changes and modifications within the spirit and scope of the invention will become apparent to those skilled in the art from this detailed description. In further embodiments, features from specific embodiments may be combined with features from other embodiments. For example, features from one embodiment may be combined with features from any of the other embodiments. In further embodiments, additional features may be added to the specific embodiments described herein.

### BRIEF DESCRIPTION OF THE DRAWINGS

For a more complete understanding, reference is now made to the following descriptions taken in conjunction with the accompanying drawings, in which:

FIG. 1 shows a schematic diagram of a fixed bed dehydrogenation reactor, according to embodiments of the invention;

FIG. 2 shows a schematic diagram of a fixed reactor bed with improved heat distribution and improved flow distribution, according to embodiments of the invention; and

FIG. 3 shows a schematic flowchart of a method of dehydrogenating isobutane to form isobutylene, according to embodiments of the invention.

### DETAILED DESCRIPTION OF THE INVENTION

A method has been discovered for dehydrogenating isobutane to isobutylene. By using a first material adapted to improve flow distribution of the fixed reactor bed, a second material adapted to improve heat distribution of the fixed reactor bed, and a third material to improve both the flow distribution and heat distribution of the fixed reactor bed, the coke formation on the catalyst can be suppressed and the catalyst life can be improved, thereby increasing conversion rate of the isobutane and reducing the operating costs for isobutane dehydrogenation.

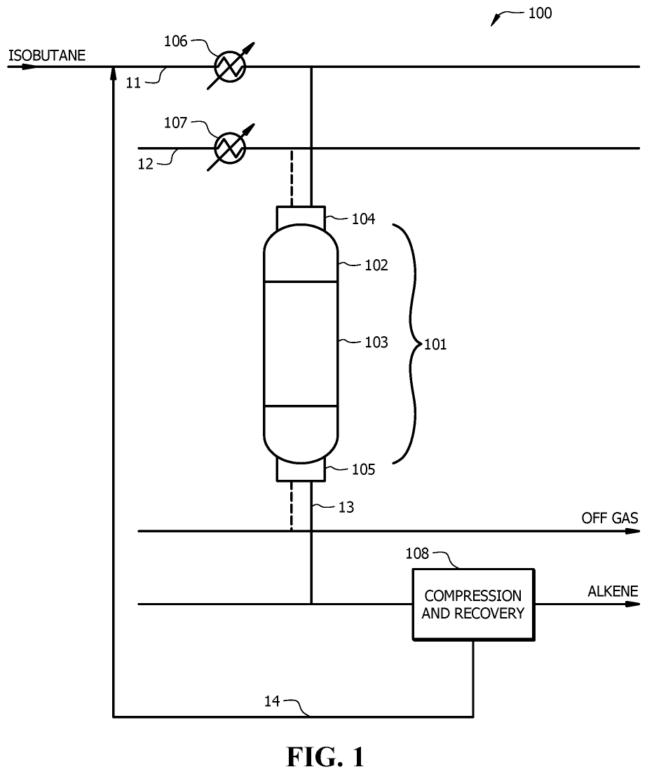

With reference to FIG. 1, a schematic diagram is shown of fixed bed dehydrogenation unit 100 for dehydrogenating isobutane to form isobutylene, according to embodiments of the invention. Dehydrogenation unit 100 may include fixed bed reactor 101. In embodiments of the invention, fixed bed reactor 101 may include reactor shell 102. Reactor shell 102 may be cylindrical. According to embodiments of the invention, fixed bed reactor 101 may include fixed reactor bed 103 disposed in reactor shell 102. Fixed bed reactor 101 may further include hydrocarbon inlet 104 disposed on reactor shell 102. Hydrocarbon inlet 104 may be configured to receive hydrocarbon feed stream 11 into reactor shell 102 during a reaction run. According to embodiments of the invention, hydrocarbon inlet 104 may be further configured to receive regeneration gas stream 12 into reactor shell 102 during catalyst regeneration.

In embodiments of the invention, fixed bed reactor 101 may further include outlet 105 disposed on reactor shell 102 on an opposite side to hydrocarbon inlet 104. Outlet 105 may be configured to discharge product stream 13 during a reaction run. Outlet 105 may be further configured to discharge an off gas from catalyst regeneration. In embodiments of the invention, hydrocarbon of hydrocarbon feed stream 11 may include isobutane. Regeneration gas stream 12 may include oxygen and/or air. In embodiments of the invention, regeneration gas stream 12 may further comprise a diluent, where different concentration of the diluent causes different concentrations of oxygen. In embodiments of the invention, the diluent may include nitrogen, steam, flue gas, or combinations thereof.

According to embodiments of the invention, dehydrogenation unit 100 may further include feed heater 106 disposed upstream of hydrocarbon inlet 104. Feed heater 106 may be configured to heat hydrocarbon feed stream 11 to a reaction temperature. Dehydrogenation unit 100 may further include regeneration gas heater 107 configured to heat regeneration gas stream 12 to a regeneration temperature. In embodiments of the invention, compression and recovery unit 108 may be disposed downstream of outlet 105. Compression and recovery unit 108 may be configured to separate product stream 13 into purified alkene (isobutylene) and recycle stream 14, which comprises unreacted hydrocarbon (isobutane).

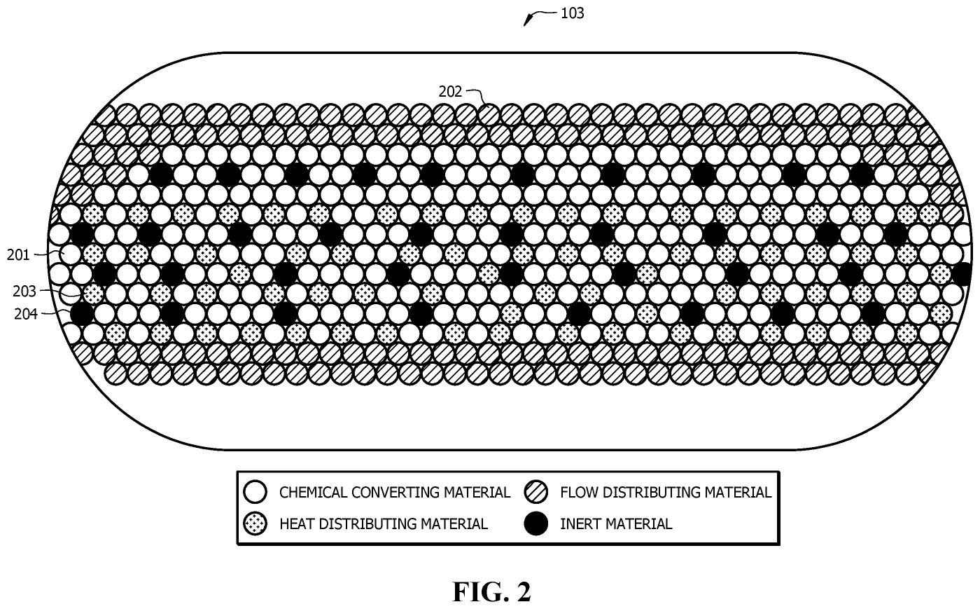

FIG. 2 shows a schematic diagram of fixed reactor bed 103. In embodiments of the invention, fixed reactor bed 103 may include catalyst 201 (chemical converting material) adapted to accelerate dehydrogenation of the isobutane to isobutylene. Catalyst 201 may include platinum, platinum-tin, chromium oxide, or combinations thereof. In embodiments of the invention, catalyst 201 may be supported on a support material comprising alumina, silica, carbon, or combinations thereof.

According to embodiments of the invention, fixed reactor bed 103 may further include first material 202 (flow distributing material) adapted to improve flow distribution such that a time difference for hydrocarbon feed stream 11 flowing between a center and an edge of fixed bed reactor 101 at a planar cross-section is in a range of 0.1 to 10 seconds. Preferably, the time difference for the hydrocarbon feed stream flowing between the center and the edge of the reactor at a planar cross-section is in a range of 1 to 5 seconds, and all ranges and values therebetween including 1 second, 2 seconds, 3 seconds, 4 seconds, and 5 seconds.

In embodiments of the invention, first material 202 may include a solid thermally stable inert material. Exemplary solid thermally stable inert materials may include, but are not limited to oxides or carbides of Al (Aluminum), Si (Silicon), Ti (Titanium), Zr (Zirconium), Zn (Zinc), Ce (Cerium), Mg (Magnesium), Ca (Calcium), La (Lanthanum), Cs (Cesium), Ba (Barium). The solid of the aforementioned oxides or carbides may be in a shape that is substantially spherical, cylindrical, ring-shaped, irregular shaped, or combinations thereof. In embodiments of the invention, first material 202 may have a particle size in a range of 5 to 35 mm, and all ranges and values therebetween including ranges of 5 to 7 mm, 7 to 9 mm, 9 to 11 mm, 11 to 13 mm, 13 to 15 mm, 15 to 17 mm, 17 to 19 mm, 19 to 21 mm, 21 to 23 mm, 23 to 25 mm, 25 to 27 mm, 27 to 29 mm, 29 to 31 mm, 31 to 33 mm, and 33 to 35 mm. In embodiments of the invention, particles of first material 202 may be substantially spherically shaped. According to embodiments of the invention, first material 202 may have a heat conductivity in a range of 0.05 to 50 W/m/K and all ranges and values therebetween including ranges of 0.05 to 0.10 W/m/K, 0.10 to 0.20 W/m/K, 0.20 to 0.30 W/m/K, 0.30 to 0.40 W/m/K, 0.40 to 0.50 W/m/K, 0.50 to 0.60 W/m/K, 0.60 to 0.70 W/m/K, 0.70 to 0.80 W/m/K, 0.80 to 0.90 W/m/K, 0.90 to 1.0 W/m/K, 1.0 to 5 W/m/K, 5 to 10 W/m/K, 10 to 15 W/m/K, 15 to 20 W/m/K, 20 to 25 W/m/K, 25 to 30 W/m/K, 30 to 35 W/m/K, 35 to 40 W/m/K, 40 to 45 W/m/K, and 45 to 50 W/m/K. In embodiments of the invention, first material 202 may have an absolute porosity of 0 to 0.3(−) and all ranges and values therebetween, including 0.05 to 0.10, 0.10 to 0.15, 0.15 to 0.20, 0.20 to 0.25, and 0.25 to 0.30.

According to embodiments of the invention, fixed reactor bed 103 may further include second material 203 (heat distributing material) adapted to improved heat distribution in fixed reactor bed 103 such that a difference between a temperature at a first location and a temperature at a second location in the reactor bed does not exceed 50° C. In embodiments of the invention, second material 203 may include a conductive material and/or an insulating material. Non-limiting examples of the conductive material may include metals or oxides of Al (Aluminum), Si (Silicon), Ti (Titanium), Zr (Zirconium), Zn (Zinc), Ce (Cerium), Mg (Magnesium), Ca (Calcium), La (Lanthanum), Cs (Cesium), Ba (Barium), Cu (Copper), Au (Gold), Sn (Tin) Fe (Iron), W (Tungsten), Ni (Nickel), Co (Cobalt), alloys thereof, and combinations thereof. In embodiments of the invention, non-limiting examples of the insulating material may include oxides or carbides of (Aluminum), Si (Silicon), Ti (Titanium), Zr (Zirconium), Zn (Zinc), Ce (Cerium), Mg (Magnesium), Ca (Calcium), La (Lanthanum), Cs (Cesium), Ba (Barium), and combinations thereof. According to embodiments of the invention, the conductive material and the insulating material may be substantially spherical, cylindrical, ring-shaped, irregular-shaped, grain shaped, or combinations thereof. In some more particular embodiments, examples for second material 203 may include but are not limited to alumina-based spheres, cylinders, rings, irregular shapes, grains, and combinations thereof.

In embodiments of the invention, second material 203 may be adapted to maintain heat for at least 8 minutes (i.e. the temperature drop is less than 40° C. within at least 8 minutes). According to embodiments of the invention, second material 203 may have a particle size of 2 to 15 mm and all ranges and values therebetween including 2 to 3 mm, 3 to 4 mm, 4 to 5 mm, 5 to 6 mm, 6 to 7 mm, 7 to 8 mm, 8 to 9 mm, 9 to 10 mm, 10 to 11 mm, 11 to 12 mm, 12 to 13 mm, 13 to 14 mm, and 14 to 15 mm. Particles of second material 203 may be in a substantially cylindrical shape. In embodiments of the invention, second material 203 may have a thermal conductivity in a range of 0.4 to 200 W/m/K and all ranges and values therebetween, including 0.4 to 0.5 W/m/K, 0.5 to 0.6 W/m/K, 0.6 to 0.7 W/m/K, 0.7 to 0.8 W/m/K, 0.8 to 0.9 W/m/K, 0.9 to 1.0 W/m/K, 1.0 to 10 W/m/K, 10 to 20 W/m/K, 20 to 30 W/m/K, 30 to 40 W/m/K, 40 to 50 W/m/K, 50 to 60 W/m/K, 60 to 70 W/m/K, 70 to 80 W/m/K, 80 to 90 W/m/K, 90 to 100 W/m/K, 100 to 110 W/m/K, 110 to 120 W/m/K, 120 to 130 W/m/K, 130 to 140 W/m/K, 140 to 150 W/m/K, 150 to 160 W/m/K, 160 to 170 W/m/K, 170 to 180 W/m/K, 180 to 190 W/m/K, and 190 to 200 W/m/K. An absolute porosity of second material 203 may be in a range of 0 to 0.5(−) and all ranges and values therebetween including 0.05 to 0.10, 0.10 to 0.15, 0.15 to 0.20, 0.20 to 0.25, 0.25 to 0.30, 0.30 to 0.35, 0.35 to 0.40, 0.40 to 0.45, and 0.45 to 0.50.

According to embodiments of the invention, fixed reactor bed 103 may further include third material 204 (inert material) that is inert with respect to the dehydrogenation of the isobutane to isobutylene. In embodiments of the invention, third material 204 may include a non-reactive material that is adapted to increase flow distribution and heat distribution in fixed reactor bed 103. No-limiting examples of third material 204 may include oxides or carbides of (Aluminum), Si (Silicon), Ti (Titanium), Zr (Zirconium), Zn (Zinc), Ce (Cerium), Mg (Magnesium), Ca (Calcium), La (Lanthanum), Cs (Cesium), Ba (Barium), and combinations thereof. Third material 204 may be substantially spherical, cylindrical, ring-shaped, irregular-shaped, grain-shaped or combinations thereof. In some more particular embodiments, examples of third material 204 may include, but are not limited to, alumina spheres, alumina cylinders, alumina rings, alumina irregular shapes, and combinations thereof.

In embodiments of the invention, catalyst 201 may form a catalyst layer. First material 202 adapted to improve flow distribution of fixed reactor bed 103 may form a top layer and a bottom layer disposed on top and bottom of the catalyst layer, respectively. According to embodiments of the invention, the top layer of first material 202 may extend to cover a top portion of a side surface of the catalyst layer. According to embodiments of the invention, particles of second material 203 adapted to improve heat distribution of fixed reactor bed 103 may be substantially evenly distributed in the catalyst layer. In embodiments of the invention, particles of third material 204 may be substantially evenly distributed in the catalyst layer.

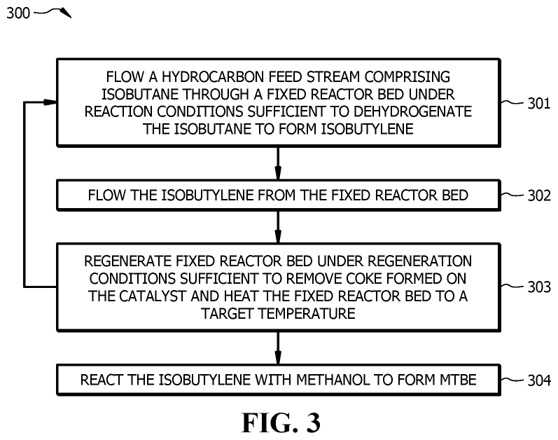

FIG. 3 shows method 300 for dehydrogenating isobutane to form isobutylene, according to embodiments of the invention. Method 300 may be implemented by dehydrogenation unit 100, as shown in FIG. 1 and fixed reactor bed 103 as shown in FIG. 2. In embodiments of the invention, as shown in Block 301, method 300 may include flowing hydrocarbon feed stream 11 comprising the isobutane through fixed reactor bed 103 under reaction conditions sufficient to dehydrogenate the isobutane to form the isobutylene.

In embodiments of the invention, the reaction conditions at block 301 may include a reaction temperature in a range of 500 to 700° C. and all ranges and values therebetween, including 500 to 510° C., 510 to 520° C., 520 to 530° C., 530 to 540° C., 540 to 550° C., 550 to 560° C., 560 to 570° C., 570 to 580° C., 580 to 590° C., 590 to 600° C., 600 to 610° C., 610 to 620° C., 620 to 630° C., 630 to 640° C., 640 to 650° C., 650 to 660° C., 660 to 670° C., 670 to 680° C., 680 to 690° C., and 690 to 700° C. The reaction conditions at block 301 may further include a reaction pressure in a range of 0.02 to 0.9 bar and all ranges and values therebetween, including the ranges of 0.02 to 0.03 bar, 0.03 to 0.04 bar, 0.04 to 0.05 bar, 0.05 to 0.06 bar, 0.06 to 0.07 bar, 0.07 to 0.08 bar, 0.08 to 0.09 bar, 0.09 to 0.10 bar, 0.10 to 0.20 bar, 0.20 to 0.30 bar, 0.30 to 0.40 bar, 0.40 to 0.50 bar, 0.50 to 0.60 bar, 0.60 to 0.70 bar, 0.70 to 0.80 bar, and 0.80 to 0.90 bar. The reaction conditions at block 301 may further include a gas hourly space velocity of hydrocarbon feed stream 11 in a range of 0.3 to 1.2 hr⁻¹ and all ranges and values therebetween including 0.3 hr⁻¹, 0.4 hr⁻¹, 0.5 hr⁻¹, 0.6 hr⁻¹, 0.7 hr⁻¹, 0.8 hr⁻¹, 0.9 hr⁻¹, 1.0 hr⁻¹, 1.1 hr⁻¹, 1.2 hr⁻¹, and 1.3 hr⁻¹. According to embodiments of the invention, method 300 may further include flowing the isobutylene from fixed reactor bed 103, as shown in block 302. The isobutylene flowed from fixed reactor bed 103 may be flowed to compression and recovery unit 108 to form purified isobutylene. In embodiments of the invention, a conversion rate of isobutane to isobutylene in block 301 may be in a range of 45 to 60% and all ranges and values therebetween, including 45 to 46%, 46 to 47%, 47 to 48%, 48 to 49%, 49 to 50%, 50 to 51%, 51 to 52%, 52 to 53%, 53 to 54%, 54 to 55%, 55 to 56%, 56 to 5′7%, 57 to 58%, 58 to 59%, or 59 to 60%.

In embodiments of the invention, method 300 may further include regenerating fixed reactor bed 102 under regeneration conditions sufficient to remove coke formed on catalyst 201 and heat fixed reactor bed 103 to a target temperature, as shown in block 303. In embodiments of the invention, the target temperature may be in a range of 500 to 700° C. and all ranges and values therebetween including ranges of 500 to 510° C., 510 to 520° C., 520 to 530° C., 530 to 540° C., 540 to 550° C., 550 to 560° C., 560 to 570° C., 570 to 580° C., 580 to 590° C., 590 to 600° C., 600 to 610° C., 610 to 620° C., 620 to 630° C., 630 to 640° C., 640 to 650° C., 650 to 660° C., 660 to 670° C., 670 to 680° C., 680 to 690° C., and 690 to 700° C. According to embodiments of the invention, the regenerating may include flowing a regeneration gas through fixed reactor bed 103. Examples of the regeneration gas may include, but are not limited to air, oxygen, a fuel, and combinations thereof. The regeneration conditions may include a temperature of regeneration gas in a range of 550 to 750° C. and all ranges and values therebetween, including ranges of 550 to 560° C., 560 to 570° C., 570 to 580° C., 580 to 590° C., 590 to 600° C., 600 to 610° C., 610 to 620° C., 620 to 630° C., 630 to 640° C., 640 to 650° C., 650 to 660° C., 660 to 670° C., 670 to 680° C., 680 to 690° C., 690 to 700° C., 700 to 710° C., 710 to 720° C., 720 to 730° C., 730 to 740° C., and 740 to 750° C. The regeneration conditions may further include regeneration pressure of 0.3 to 2.5 bar and all ranges and values therebetween, including 0.3 to 0.4 bar, 0.4 to 0.5 bar, 0.5 to 0.6 bar, 0.6 to 0.7 bar, 0.7 to 0.8 bar, 0.8 to 0.9 bar, 0.9 to 1.0 bar, 1.0 to 1.1 bar, 1.1 to 1.2 bar, 1.2 to 1.3 bar, 1.3 to 1.4 bar, 1.4 to 1.5 bar, 1.5 to 1.6 bar, 1.6 to 1.7 bar, 1.7 to 1.8 bar, 1.8 to 1.9 bar, 1.9 to 2.0 bar, 2.0 to 2.1 bar, 2.1 to 2.2 bar, 2.2 to 2.3 bar, 2.3 to 2.4 bar, and 2.4 to 2.5 bar. A gas hourly space velocity for the regeneration gas at block 303 may be in a range of 0.3 to 5 hr⁻¹ per hour and all ranges and values therebetween, including 0.3 hr⁻¹, 0.4 hr⁻¹, 0.5 hr⁻¹, 0.6 hr⁻¹, 0.7 hr⁻¹, 0.8 hr⁻¹, 0.9 hr⁻¹, 1.0 hr⁻¹, 1.1 hr⁻¹, 1.2 hr⁻¹, 1.3 hr⁻¹, 1.4 hr⁻¹, 1.5 hr⁻¹, 1.6 hr⁻¹, 1.7 hr⁻¹, 1.8 hr⁻¹, 1.9 hr⁻¹, 2.0 hr⁻¹, 2.1 hr⁻¹, 2.2 hr⁻¹, 2.3 hr⁻¹, 2.4 hr⁻¹, 2.5 hr⁻¹, 2.6 hr⁻¹, 2.7 hr⁻¹, 2.8 hr⁻¹, 2.9 hr⁻¹, 3.0 hr⁻¹, 3.1 hr⁻¹, 3.2 hr⁻¹, 3.3 hr⁻¹, 3.4 hr⁻¹, 3.5 hr⁻¹, 3.6 hr⁻¹, 3.7 hr⁻¹, 3.8 hr⁻¹, 3.9 hr⁻¹, 4.0 hr⁻¹, 4.1 hr⁻¹, 4.2 hr⁻¹, 4.3 hr⁻¹, 4.4 hr⁻¹, 4.5 hr⁻¹, 4.6 hr⁻¹, 4.7 hr⁻¹, 4.8 hr⁻¹, and 4.9 hr⁻¹. According to embodiments of the invention, blocks 301 to 303 may be repeated.

As described above, fixed reactor bed 103 may include first material 202 adapted to improve flow distribution in fixed reactor bed 103, second material 203 adapted to improve heat distribution in fixed reactor bed 103, and third material 204 adapted to improve both flow distribution and heat distribution in fixed reactor bed 103. In embodiments of the invention, method 300 may not include injection of sulfur in fixed reactor bed 103, thereby eliminating the sulfur contamination in the alkene product (e.g. isobutylene). Furthermore, in embodiments of the invention, catalyst 201 may have a catalyst life of 6 to 48 months and all ranges and values therebetween including 6 to 12 months, 12 to 18 months, 18 to 24 months, 24 to 30 months, 30 to 36 months, 36 to 42 months, and 42 to 48 months. A reaction duration for block 301 may be in a range of 4 to 12 minutes and all ranges and values therebetween including 5 minutes, 6 minutes, 7 minutes, 8 minutes, 9 minutes, 10 minutes, and 11 minutes. Blocks 301 to 303 may be repeated for at least 55,000 cycles. In embodiments of the invention, method 300 may include reacting the isobutylene with methanol to form methyl tertiary butyl ether (MTBE), as shown in block 304.

In summary, embodiments of the invention involve systems and methods for dehydrogenating isobutane to form isobutylene. The fixed reactor bed in the system may include a first material to improve flow distribution in the fixed reactor bed, a second material adapted to improve thermal distribution of the fixed reactor bed, and an inert material adapted to improve both flow distribution and thermal distribution of the fixed reactor bed. By disposing the first material on both top, bottom and side surface of the catalyst layer of the fixed reactor bed, the flow distribution in the fixed reactor bed can be improved. By evenly distributing the second material and the inert material through the catalyst layer, the thermal distribution in the fixed bed reactor can be improved and flow distribution in the fixed reactor bed can be further improved, thereby improving the conversion rate of isobutane and increasing catalyst life of fixed reactor bed.

Although embodiments of the present invention have been described with reference to blocks of FIG. 3, it should be appreciated that operation of the present invention is not limited to the particular blocks and/or the particular order of the blocks illustrated in FIG. 3. Accordingly, embodiments of the invention may provide functionality as described herein using various blocks in a sequence different than that of FIG. 3.

Although embodiments of the present application and their advantages have been described in detail, it should be understood that various changes, substitutions and alterations can be made herein without departing from the spirit and scope of the embodiments as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the above disclosure, processes, machines, manufacture, compositions of matter, means, methods, or steps, presently existing or later to be developed that perform substantially the same function or achieve substantially the same result as the corresponding embodiments described herein may be utilized. Accordingly, the appended claims are intended to include within their scope such processes, machines, manufacture, compositions of matter, means, methods, or steps.

1. A method of dehydrogenating isobutane (C₄H₁₀) to isobutylene (C₄H₈), the method comprising:
flowing a hydrocarbon feed stream comprising the isobutane through a fixed reactor bed under reaction conditions sufficient to dehydrogenate the isobutane to the isobutylene, wherein the fixed reactor bed comprises:
a catalyst adapted to accelerate dehydrogenation of the isobutane to the isobutylene;
a first material adapted to improve flow distribution such that a time difference for the hydrocarbon feed stream flowing between a center and an edge of the reactor at a planar cross-section is in a range of 0.1 to 10 seconds; and
a second material adapted to improve heat distribution such that a difference between a temperature at a first location and a temperature at a second location in the reactor bed does not exceed 60° C.; and
flowing the isobutylene from the fixed reactor bed.

2. The method of claim 1, further comprising reacting the isobutylene with methanol to form methyl tertiary butyl ether (MTBE).

3. The method of claim 1, wherein the reaction conditions comprise a reaction temperature in a range of 500 to 700° C.

4. The method of claim 1, wherein the reaction conditions comprise a reaction pressure in a range of 0.02 to 0.9 bar.

5. The method of claim 1, wherein the catalyst is selected from the group consisting of chromium oxide, platinum, platinum-tin, and combinations thereof.

6. The method of claim 1, wherein the time difference for the hydrocarbon feed stream flowing between the center and the edge of the reactor at a planar cross-section is in a range of 1 to 5 seconds.

7. The method of claim 1, wherein the first material adapted to improve flow distribution comprises a solid thermally stable inert material premixed with the second material and the third materials in the fixed reactor bed.

8. The method of claim 7, wherein the thermally stable inert material is selected from the group consisting of oxides or carbides of Al, Si, Ti, Zr, Zn, Ce, Mg, Ca, La, Cs, Ba, and combinations thereof.

9. The method of claim 1, wherein the first material has a particle size of 5 to 35 mm and a geometric shape of substantially spherical (shape).

10. The method of claim 1, wherein the first material has a thermal conductivity in a range of 0.05 to 5 W/m/K, and an absolute porosity of 0 to 0.3(−).

11. The method of claim 1, wherein the second material adapted to improve heat distribution comprises a conductive material and/or an insulating material.

12. The method of claim 11, wherein the conductive material is selected from the group consisting of metal or oxides of Al, Si, Ti, Zr, Zn, Ce, Mg, Ca, La, Cu, Au, Sn, Fe, W, Ni, Co, Cs, Ba, alloys thereof, and combinations thereof and the insulating material is selected from the group consisting of oxides or carbides of Al, Si, Ti, Zr, Zn, Ce, Mg, Ca, La, Cs, Ba, and combinations thereof.

13. The method of claim 1, wherein the second material is adapted to maintain a temperature drop thereof less than 40° C. within at least 8 minutes.

14. The method of claim 1, wherein the second material has a particle size of 2 to 15 mm and a geometric shape of substantially cylindrical (shape).

15. The method of claim 1, wherein the fixed reactor bed further comprises a third material that is inert with respect to the dehydrogenating of the isobutane to the isobutylene.

16. The method of claim 1, wherein the third material comprises a non-reactive material that is adapted to increase flow distribution and heat distribution in the fixed reactor bed.

17. The method of claim 16, wherein the third material includes oxides or carbides of Al, Si, Ti, Zr, Zn, Ce, Mg, Ca, La, Cs, Ba, or combinations thereof.

18. The method of claim 1, wherein the isobutane has a conversion rate of 45 to 60%.

19. The method of claim 1, wherein the flowing the hydrocarbon feed stream does not include injecting sulfur to the fixed reactor bed.

20. A fixed bed reactor for dehydrogenating a hydrocarbon, the fixed bed reactor comprising:
a reactor shell;
a fixed reactor bed comprising a catalyst adapted to accelerate dehydrogenation of the isobutane to the isobutylene;
a first material adapted to improve flow distribution such that a time difference for the hydrocarbon feed stream flowing between a center and an edge of the reactor at a planar cross-section is in a range of 0.1 to 10 seconds; and
a second material adapted to improve heat distribution such that a difference between a temperature at a first location and a temperature at a second location in the reactor bed does not exceed 60° C.;
a hydrocarbon inlet disposed on a side of the reactor shell, wherein the hydrocarbon inlet is adapted to receive a hydrocarbon feed stream and/or a regeneration gas into the reactor shell; and
an outlet disposed on a side of the reactor shell that is opposite to the side that hydrocarbon inlet is disposed on, wherein the outlet is adapted to discharge a product stream from the reactor shell.
